(19) United States
(12) Patent Application Publication
(10) Pub. No.: US 2010/0186741 A1
(43) Pub. Date: Jul. 29, 2010

(54) Method and System for Detecting Mouth Leak During Application of Positive Airway Pressure

(76) Inventors: Alonzo C. Aylsworth, Wildwood, MO (US); Charles R. Aylsworth, Wildwood, MO (US); Lawrence C. Spector, Austin, TX (US)

Correspondence Address: DUBOIS, BRYANT, CAMPBELL & SCHWARTZ, LLP, 700 LAVACA STREET, SUITE 1300, AUSTIN, TX, 78701, US

(21) Appl. No.: 12/696,722

(22) Filed: Jan. 29, 2010

Related U.S. Application Data
(60) Provisional application No. 61/148,088, filed on Jan. 29, 2009.

(51) Int. Cl. A61M 16/00; A61M 16/16

(52) U.S. Cl. 128/203.29; 128/204.23

(57) ABSTRACT

A method and system for providing therapeutic gas to a patient during positive airway pressure ventilation and, more particularly, detecting the presence of a mouth leak during ventilation and, upon the detection of a mouth leak, reducing the applied pressure so as to reduce irritation and discomfort experienced by the patient. Respiratory air flow from a patient is measured in a waveform as a function of time. An approximate value of the root mean square voltage of the waveform is established during a period in which the patient is experiencing a mouth leak and a root mean square voltage of the waveform is established during a period in which the patient is experiencing an apneic event. The waveform is subsequently monitored and the rate of respiratory airflow is decreased when there is an indication of a mouth leak provided there is no indication of an apneic event.

### PRIORITY STATEMENT UNDER 35 U.S.C. §119 & 37 C.F.R. §1.78

This non-provisional application claims priority based upon prior U.S. Provisional Patent Application Ser. No. 61/148,088 filed Jan. 29, 2009 in the name of Alonzo C. Aylsworth, Charles R. Aylsworth and Lawrence C. Spector entitled “Method and System Responsive to Detecting Mouth Leak in Application of Positive Airway Pressure,” the disclosure of which is incorporated herein in its entirety by reference.

### BACKGROUND OF THE INVENTION

During normal sleep patterns, air enters a patient's nares, passes the genioglossus throat muscle, and flows down into the respiratory tract and into the lungs, thereby contributing to patient's ventilation. In some patients, the genioglossus throat muscle relaxes during sleep. When this occurs, the relaxed muscle can partially or completely block the patient's airway resulting in disturbed breathing, snoring and obstructive sleep apnea.

As shown in FIG. 1, in the case of obstructive sleep apnea, the patient experiences repetitive episodes of pharyngeal (upper) airway 101 collapse or narrowing during sleep. The pharyngeal muscles relax during sleep and gradually allow the pharynx 102 to collapse. Collapse of the pharyngeal airway can block airflow or significantly restrict airflow, resulting in hypopnea. An episode of apnea or hypopnea is interrupted by a brief arousal or a lighter stage of sleep, accompanied by activation of the upper airway dilator muscles and restoration of airway patency. This cycle can occur repeatedly throughout the patient's sleep.

To treat obstructive sleep apnea, continuous positive airway pressure (CPAP) systems continuously impose a positive airway pressure on the patient's airways. This positive air pressure assists in maintaining positive pressure within the patient's airway, thereby maintaining airway patency. Pressurized air or gas is typically supplied to the respiratory system through a full face mask, a nasal mask or nasal cannulae. Nasal masks have become popular, in part because less of the face has to be covered than with a full face mask.

In some cases, pressurized air flows through the velopharyngeal sphincter (i.e. between the lateral pharyngeal walls and the soft palate) into the oral cavity and then out through the lips, resulting in a mouth leak. When a mouth leak occurs, pressurized air does not reach the lungs and does not contribute to ventilation, thereby rendering the treatment less effective or ineffective. In addition, because of the one-way airflow through the nasal passages, mouth leaks tend to dry the mucosal surfaces resulting in nasal congestion after only several hours of use. In some applications, the CPAP system will apply a higher pressure through the nose mask when a mouth or mask leak is detected to compensate for the leak which only exacerbates the problem. In many cases, the side effects are often so severe that the patient is no longer able to tolerate treatment.

In some cases, CPAP machines humidify the air before it is supplied to the nares of the patient. Humidifying the air may help reduce nasal irritation. For the reasons described above, airflow escaping through the mouth flows at a much higher velocity than air that is properly directed through the respiratory tract. As a result, a mouth leak can lower the relative humidity of the therapeutic airstream and further promote nasal irritation.

As previously discussed, obstructive sleep apnea can occur intermittently. Many patients do not have obstructive sleep apnea throughout the night. Patients have been observed during CPAP therapy breathing normally with their mouth open, yet the CPAP machine will unsuccessfully continue to attempt therapy, blowing CPAP airflow continuously through their nose and out of their mouth.

Various methods have been employed to address the reduction or elimination of mouth leaks during CPAP treatment. For example, a mask known in the art is shown in FIG. 2A. Mask 10 comprises a nose portion 12 which covers the nose, and a seal 14 which seals against the patient's face to allow a greater pressure within the cavity 16 of the nose portion 12. The nose portion 12 fluidly couples to a hose portion 18 which fluidly couples to a source of positive pressure, such as a positive airway pressure machine. The mask 10 further comprises a sensing tube 20 that has a patient end 22 that terminates proximate to a patient's mouth. In the embodiment illustrated by FIG. 2A, air escaping from the mouth is hydraulically forced into the sensing tube 20, and therefore attributes of airflow indicative of air leaks through the patient's mouth may be sensed by pressure and/or flow sensor on a device end of the sensing tube 20.

Referring now to FIG. 2B which shows another mask 10 known in the art. In this mask, the sensing tube 20 is configured such that air escaping the patient's mouth creates a lower pressure at patient end 22, and if the sensing tube is open to airflow this lower pressure induces airflow through the sensing tube 20 toward the patient. In this mask, the attribute of airflow indicative of air leaks from the mouth may be pressure sensed by a pressure sensor, or airflow sensed by a flow sensor.

FIG. 3 shows an elevational side view of the mask 10 of FIG. 2A on a patient 24. In particular, the nose portion 12 covers the patient's nose 26, and the seal 14 seals to the patient's face. FIG. 3 further shows the sensing tube 20 with the patient end 22 terminating proximate to the patient's mouth. Also shown in FIG. 3 is an illustrative positive airway pressure machine 28. The illustrative positive airway pressure machine 28 comprises a processor 29 electrically coupled to and controlling a fan or blower 30. The blower 30 fluidly couples to the cavity 16 of the mask 10 by way of the hose portion 18. In some cases, the positive airway pressure machine 28 comprises a flow sensor 32 fluidly coupled within the flow path between the blower 30 and the mask 10. In addition to, or in place of, the flow sensor 32, a positive airway pressure machine 28 may have a pressure sensor 34 fluidly coupled to the blower 30 and hose portion 18. When in pressure control, the blower 30 (as commanded by the processor 29) controls the pressure to a setpoint pressure using the pressure sensed by the pressure sensor 34. In other cases, the pressure applied may be proportional to the speed of the blower 30, and, thus, even when it is desirable to control pressure, a pressure sensor 34 may not be needed. In yet still other cases, the positive airway pressure machine 28 may supply a prescribed flow rate of air, substantially independent of applied pressure.

Positive airway pressure machine 28 may also comprise a sensor 36 electrically coupled to the processor 29. The sensor 36 fluidly couples to the device end 23 of sensing tubing 20 and the sensing tubing 20 senses an attribute of airflow proximate to the patient. In particular, when the patient develops a mouth leak the escaping air interacts with the patient end 22. In those cases where the sensor 36 is a flow sensor (vented to atmosphere as shown in dashed lines), the escaping air causes airflow through the sensor 36. In cases where the sensor 36 is a pressure sensor, the escaping air causes pressure fluctuations sensed by the sensor 36. When the patient end 22 is oriented as shown in FIG. 2A, escaping air causes airflow into the patient end 22, which may be sensed as airflow toward the positive airway pressure device 28 (if sensor 36 is a flow sensor), or which may be sensed as increased pressure (if sensor 36 is a pressure sensor). When the patient end 22 is oriented as shown in FIG. 2B, escaping air causes airflow out of the patient end 22, which may be sensed as airflow away from the positive airway pressure device 28 (if sensor 36 is a flow sensor), or which may be sensed as decreased pressure (if sensor 36 is a pressure sensor).

Other approaches to detecting leaks have also been described in the art. For example, certain CPAP machines algorithmically determine the presence of a mask leak at the CPAP machine end, and inform the user so that the leak can be addressed. Typically the user will be instructed to adjust their mask or will be fitted for an alternate style of mask. However, from a CPAP machine perspective, addressing the leak substantially consists of merely increasing airflow to make up for the pressure losses, or to make no changes at all, possibly leaving the patient without therapeutic benefits of positive airway therapy. Mask leaks and mouth leaks are largely seen as normal and acceptable.

Unfortunately, CPAP machines known in the art do not effectively differentiate between a mouth leak and a nasal mask leak. The one common element in all related art CPAP machines is that when a mouth leak occurs, the therapy fails. When the patient is receiving CPAP therapy, positive pressure is only available when the mouth is closed. When the mouth opens, the applied airflow and resulting pressure escape to atmosphere. With oral pressure at near atmospheric levels, the nasal CPAP airflow velocity increases dramatically through the nares. This increase in airflow velocity causes nasal irritation and results in an increase in nasal resistance. The resulting patient discomfort lowers the success rate of patient prescription compliance. The resulting increase in nasal resistance lowers the chances of successful CPAP treatment since the pressure drop, from the nasal opening where the pressure is applied, to the soft palate increases. Thus, less pressure exists in the oral airway to prevent obstructive sleep apnea.

Determination of a mouth leak verses other breathing circuit leaks using prior art techniques often fail because the position of the patient's soft palate, or genio-glossus throat muscle, is not considered. While it may be desirable to partially reduce the airflow to a patient if the patient's oral airway is partially blocked by the soft palate, this is typically not possible because conventional CPAP machines cannot detect a partial blockage.

Positive airway pressure systems often include a means for ramping from a startup pressure to a prescribed pressure. When positive airway pressure systems are auto-titrating the target pressure is defined by the pressure which provides adequate airway support and elimination of patient respiratory events within a preset pressure limit. Sleep efficiency is lost when such means are employed. Arousals may occur during the search process for the best titration pressure.

### SUMMARY OF THE INVENTION

The invention contemplates the treatment of sleep apnea through application of pressure at variance with ambient atmospheric pressure within the upper airway of the patient in a manner to promote dilation of the airway to thereby improve upper airway patency during sleep. More particularly, the present invention is concerned with a method and apparatus for detecting the presence of a mouth leak during ventilation and, upon the detection of a mouth leak, reducing the applied pressure so as to reduce irritation and discomfort experienced by the patient. In one embodiment, respiratory air flow from a patient is measured in a waveform as a function of time. An approximate value of the root mean square voltage of the waveform is established during a period in which the patient is experiencing a mouth leak and a root mean square voltage of the waveform is established during a period in which the patient is experiencing an apneic event. The waveform is subsequently monitored and the rate of respiratory airflow is decreased when there is an indication of a mouth leak provided there is no indication of an apneic event.

In other embodiments, the rate of respiratory airflow is increased when there is no longer an indication of a mouth leak or when there is an indication of an apneic event. In other embodiments, the humidity of the respiratory airflow is adjusted as the rate of respiratory airflow decreases and the humidity is readjusted as the rate of respiratory airflow increases.

In still other embodiments, an approximate value of the root mean square voltage of the waveform is established during a period in which the patient's soft palate is partially blocking the oral airway, the waveform is subsequently monitored and the rate of respiratory airflow is decreased when there is an indication of a partial blockage of the oral airway provided there is no indication of an apneic event. The reduction in airflow in response to an indication of a partial blockage of the patient's oral airway may be less than the reduction in response to an indication of a full apneic event.

The foregoing has outlined rather broadly certain aspects of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### DESCRIPTION OF THE DRAWINGS

For a more complete understanding of the present invention, and the advantages thereof, reference is now made to the following descriptions taken in conjunction with the accompanying drawings, in which:

FIG. 1 is a side view depicting the collapse of the pharyngeal airway during sleep;

FIG. 2A is an isometric view of a mask used in connection with at least some embodiments of the present invention;

FIG. 2B is another isometric view of a mask used in connection with at least some embodiments of the present invention;

FIG. 3 is an elevational side view of a mask together with a positive airway machine used in connection with at least some embodiments of the present invention;

FIG. 4 shows the airflow and pressure voltage waveforms of a patient breathing on a nasal mask while undergoing positive airway therapy;

FIG. 5 shows the airflow and pressure voltage waveforms of a patient breathing on a nasal mask while undergoing positive airway therapy;

FIG. 6 shows the airflow and pressure voltage waveforms of a patient breathing on a nasal mask while undergoing positive airway therapy;

FIG. 7 shows the airflow and pressure voltage waveforms of a patient breathing on a nasal mask while undergoing positive airway therapy;

FIG. 8 is a flow diagram showing the process for responding to apnea by resuming therapeutic airflow;

FIG. 9 is a flow diagram showing the process for responding to apnea by increasing therapeutic airflow;

FIG. 10 is a flow diagram showing the process for responding to apnea by increasing therapeutic airflow and restoring humidification settings.

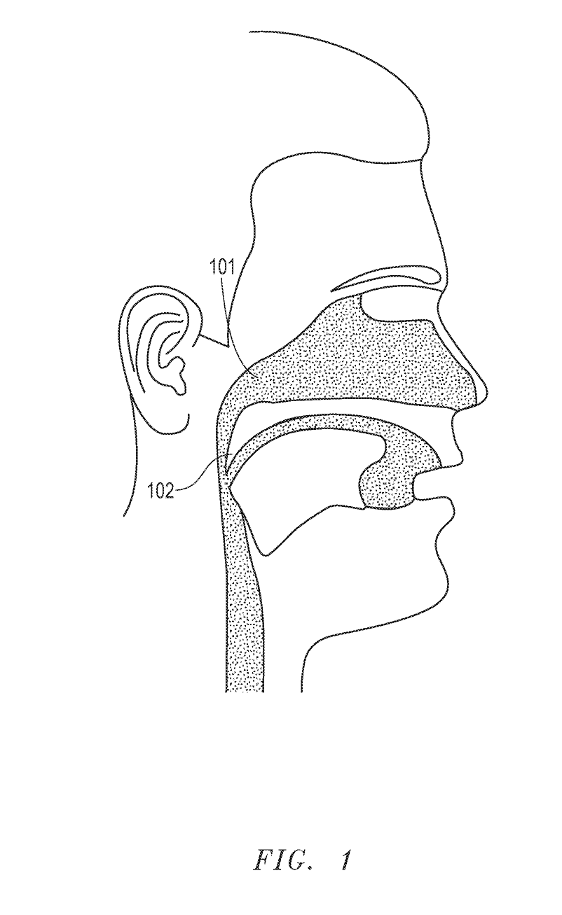

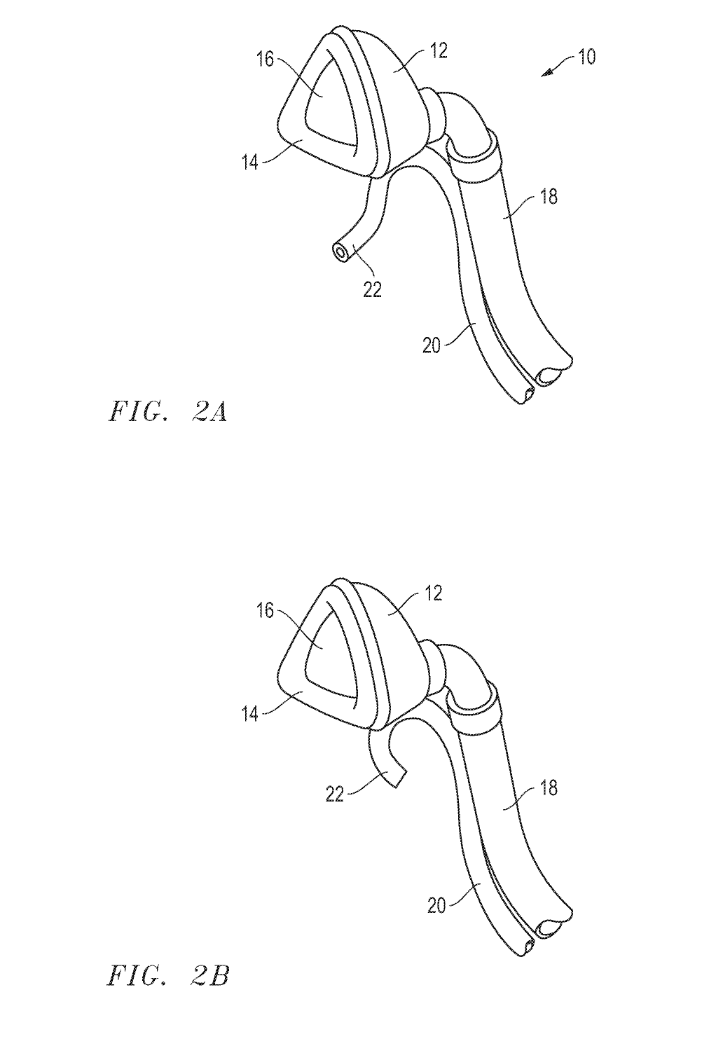

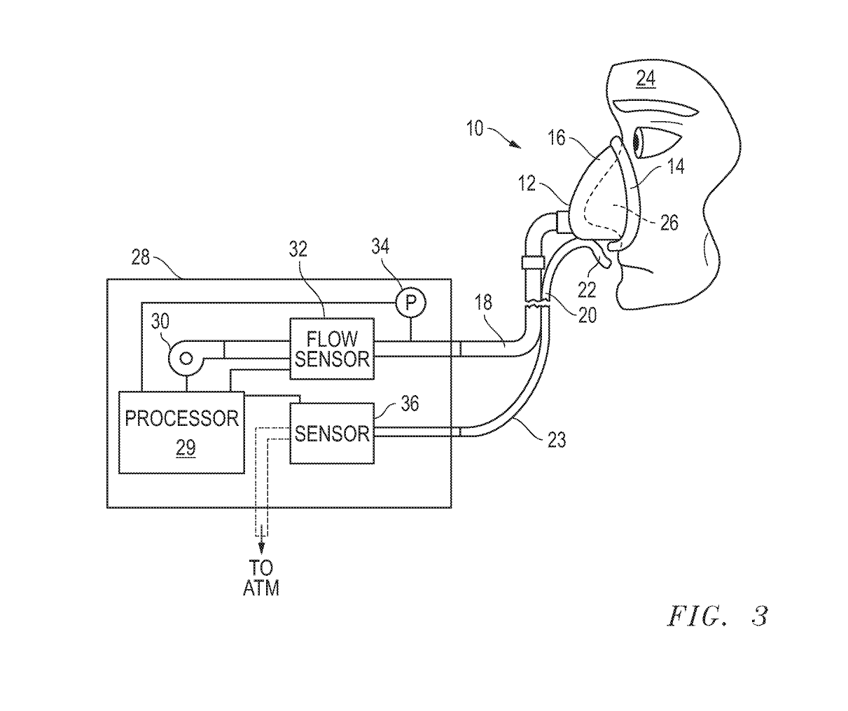

### DESCRIPTION OF THE PREFERRED EMBODIMENTS

The present invention is directed to improved methods and systems for detecting mouth leaks during the application of positive airway pressure and is particularly useful in treating disturbed breathing, snoring, obstructive sleep apnea, and certain cardiovascular sleep conditions. The configuration and use of the presently preferred embodiments are discussed in detail below. It should be appreciated, however, that the present invention provides many applicable inventive concepts that can be embodied in a wide variety of contexts other than the detection of mouth leaks. Accordingly, the specific embodiments discussed are merely illustrative of specific ways to make and use the invention, and do not limit the scope of the invention. In addition, references to the detection of mouth leaks and other terms used herein may be applicable to devices other than CPAP machines.

In various embodiments, the present invention is useful for the determination of mouth leaks, rather than nasal mask leaks, when a patient is using positive airway treatment, and provides improved therapy in situations where a mouth leak is experienced, preferably where normal breathing is occurring during the mouth leak event.

When distinguishing mouth leaks from mask leaks, it is important to determine the position of the soft palate. Apneas are generally categorized as either central, where there is no respiratory effort by the patient, or obstructive, where there is respiratory effort by the patient. With some central apneas, the airway is open, and the subject is merely not attempting to breathe. Conversely, with other central apneas, and with all obstructive apneas, the airway is closed. The occlusion is typically caused by the tongue or soft palate.

Apneas and other sleep-related occlusions of the airway are commonly treated through the application of continuous positive airway pressure. CPAP is generally administered by the provision of a positive pressure in the range of 4 to 20 cm H2O. The air is supplied by a motor driven blower through a hose to a mask which covers the nose and/or mouth or through nasal cannulae. There is typically an exhaust valve in the tube near the mask. Oxygen or other gases may be supplied as part of the CPAP treatment, all of which are commonly referred to herein as air.

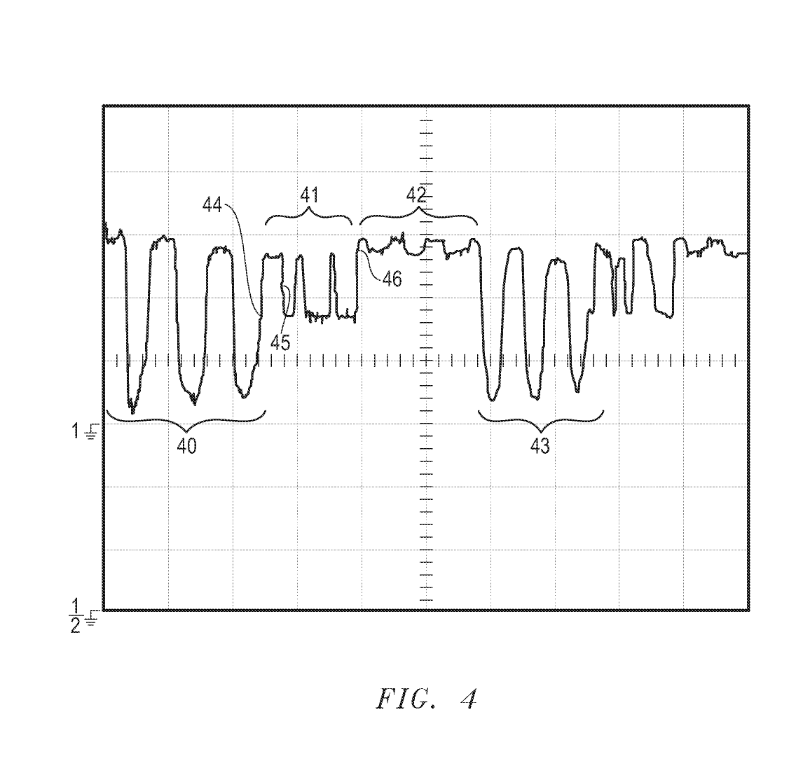

During evaluation, air flow and pressure of the air supplied to the mask can be monitored through flow and pressure sensors. The voltage waveforms of the flow-time curve provide measurable data relating to the patient's breathing patterns, the presence of obstructive sleep apnea, and the position of the soft palate. For example, FIG. 4 illustrates the voltage waveforms of a patient breathing while undergoing positive airway therapy at a pressure of 6 cm/H2O. The upper half of a waveform depicts the patient's inhalation and the lower half of the waveform depicts the patient's exhalation. For discussion purposes, various regions are identified on FIG. 4. The regions are intended to be approximate only and not intended to strictly delineate a particular event. The waveforms in region 40 indicate normal breathing with the mouth closed. The waveforms in region 41 indicate normal breathing with the mouth open, and, likewise, the waveforms in region 42 indicate normal breathing with the mouth open. The waveforms in region 43 again indicates normal breathing with the mouth closed. Inhalation tidal volume of regions 40, 41, 42, and 43 are all essentially equal. The waveforms in region 43 once again indicate normal breathing with the mouth closed.

Notice that even though the waveforms in regions 41 and 42 both indicate normal breathing with the mouth open, the waveforms are different. The waveforms in region 41 depict increased airflow measurement because the soft palate is at least partially blocking the oral airway which results in less airflow escaping to atmosphere through the patient's mouth. The waveforms in region 42 depict decreased airflow measurement because the soft palate is not blocking, or at least only partially blocking, the oral airway which results in more airflow escaping to atmosphere through the patient's mouth.

By electronically monitoring these waveforms, it is possible to determine with reasonable accuracy the airflow null voltage, defined as the root mean square (RMS) voltage of the waveform. For example, line 44 indicates the approximate null voltage for region 40. Line 45 indicates the approximate null voltage for region 41. Line 46 indicates the approximate null voltage for region 42. Thus, as indicated in FIG. 4, the RMS voltage level becomes an indicator of the amount of leak in a patient breathing circuit (i.e. the higher the null voltage, the greater the amount of air escaping through the patient's mouth).

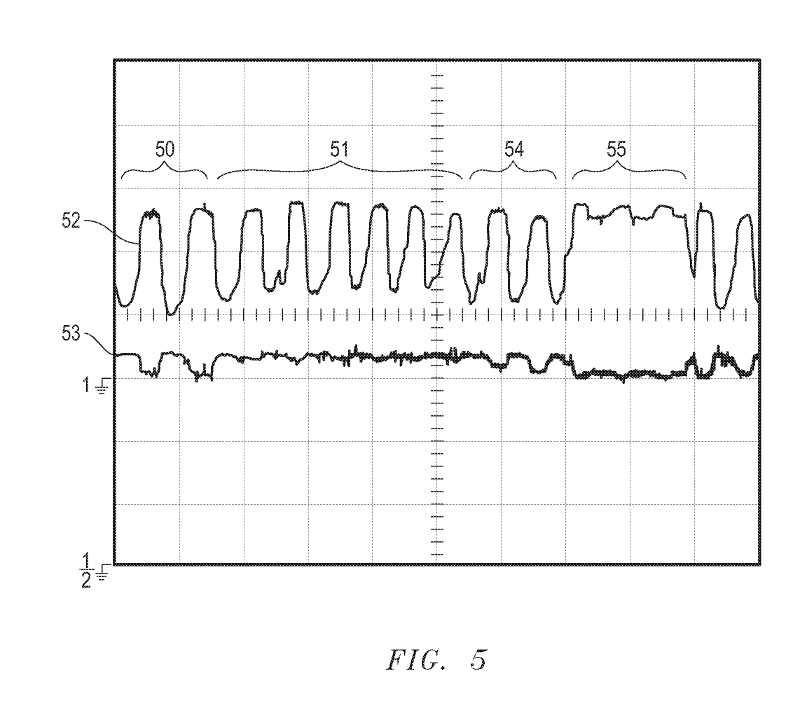

FIG. 5 illustrates the voltage waveforms of a patient breathing while undergoing positive airway therapy at a pressure of 6 cm/H2O. The waveforms in regions 50 and 54 indicate normal breathing with the mouth closed. Likewise, the waveforms in region 51 indicate normal breathing with the mouth closed but with a significant nasal mask leak. Voltage line 52 indicates airflow from the patient. Voltage line 53 indicates the patient breathing circuit pressure, roughly equivalent to the patient airway pressure at the opening of the nares. Notice that voltage line 52 in region 50 indicates a lower RMS voltage than the voltage line 52 in region 51. This is an indication of a nasal mask leak since the change in the RMS voltage is small as compared to the RMS levels indicated in FIG. 4.

Referring now to region 55 of FIG. 5. The waveforms in region 55 indicate that the patient is breathing with their mouth open. The airflow RMS voltage level is very high as compared to the nasal mask waveforms of region 51.

Additional algorithmic analyses of nasal mask leak versus mouth leak are possible by also monitoring the pressure line 53. Note that the pressure line 53 of region 50 has an RMS voltage level which is less than the RMS voltage level of region 51 where the patient is experiencing a mask leak. Additionally note that the RMS voltage level of the waveforms in region 55, where the patient is breathing with a mouth leak, is much less than the situations depicted by the waveforms in regions 50 and 51.

Another important indicator to be measured may be the peak-to-peak levels of the waveforms 52 and/or 53 to determine the type of leak, if any, experienced by the patient. It should also be appreciated that the delivery pressure to the patient will vary based upon the prescription level or levels dictated by the physician. Algorithmically comparing the RMS flow value to the actual applied pressure provides a more accurate determination of leak values. Additionally, FIG. 4 illustrates the ability of the present invention to determine the position of the soft palate during positive airway pressure therapy by, in one instance, measuring the RMS voltage and comparing that voltage to the waveform being analyzed. The information disclosed in the discussion of FIG. 4 and FIG. 5 may be processed algorithmically with common art means to quantify mouth versus nasal mask leak, and the position of the soft palate. Templates, tables, arrays, and the like may also be used for such determinations.

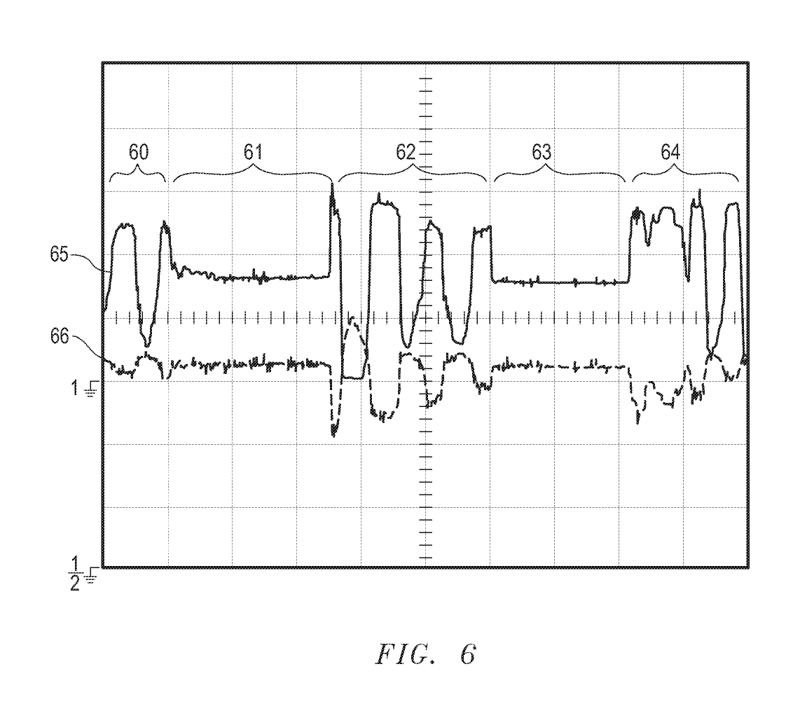

Referring now to FIG. 6 which illustrates the voltage waveforms of a patient breathing with a nasal mask while undergoing positive airway pressure therapy at a pressure of 6 cm/H2O. Line 65 is representative of the airflow delivered to the patient's breathing circuit. Line 66 is representative of the pressure delivered to the patient breathing circuit. The waveforms in region 60 indicate normal patient breathing, with no leaks. The waveforms in region 61 indicate an apnea event with no leaks. The waveforms in region 62 indicate a recovery breath with no leaks and subsequent normal breathing. The waveforms in region 63 indicate an apnea event with the patient's mouth open but the soft palate is blocking most of the airflow from escaping to atmosphere. Notice the RMS voltage levels for airflow and pressure in regions 61 and 63 are essentially identical. The waveforms in both regions indicate an apnea.

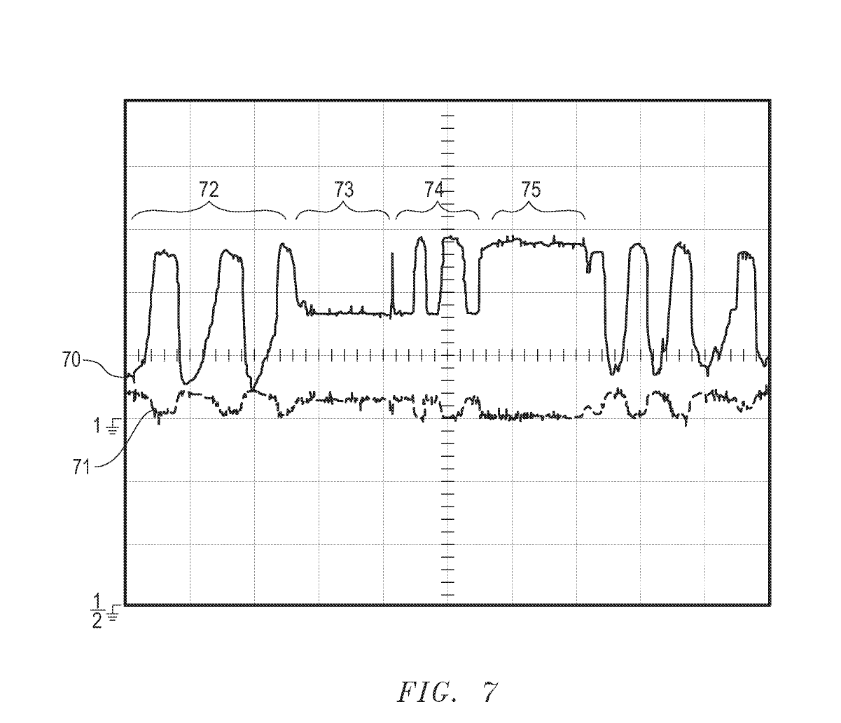

Referring now to FIG. 7 which depicts the voltage waveforms of a patient breathing on a nasal mask while undergoing positive airway therapy at a pressure of 6 cm/H2O. Airflow line 70 is representative of the airflow delivered to the patient's breathing circuit. Pressure line 71 is representative of the pressure delivered to the patient's breathing circuit. The waveforms in region 72 indicate normal patient breathing with no leaks. The waveforms in region 73 indicate an apnea event with no leaks. The waveforms in region 74 indicate an apnea event with the mouth open and the soft palate is intermittently blocking at least some of the airflow escaping from the mouth to atmosphere. The waveforms in region 74 show that it is possible to algorithmically determine the movement of the soft palate during the mouth open condition and to further determine that the apnea event is still occurring based upon the RMS voltage level of the airflow line 70, and/or based upon the RMS voltage level of the pressure line 71. In region 75, the patient still has their mouth open but the majority of the airflow is escaping to atmosphere. The patient's apnea event actually is occurring from the start of region 73 to the end of region 75.

Using these novel methods it is possible to further process algorithmically with common art means to quantify mouth versus nasal mask leak, and the position of the soft palate, and to determine and quantify apneic events. Common art devices do not consider the movement of the soft palate and as such may score such movement as normal breathing when in fact the patient may be experiencing an apnea or hypopnea event. Additionally, the flow and pressure values may similarly be used to determine and quantify hypopnea events. Common art templates, tables, arrays, and the like may also be used for such determinations using these novel methods.

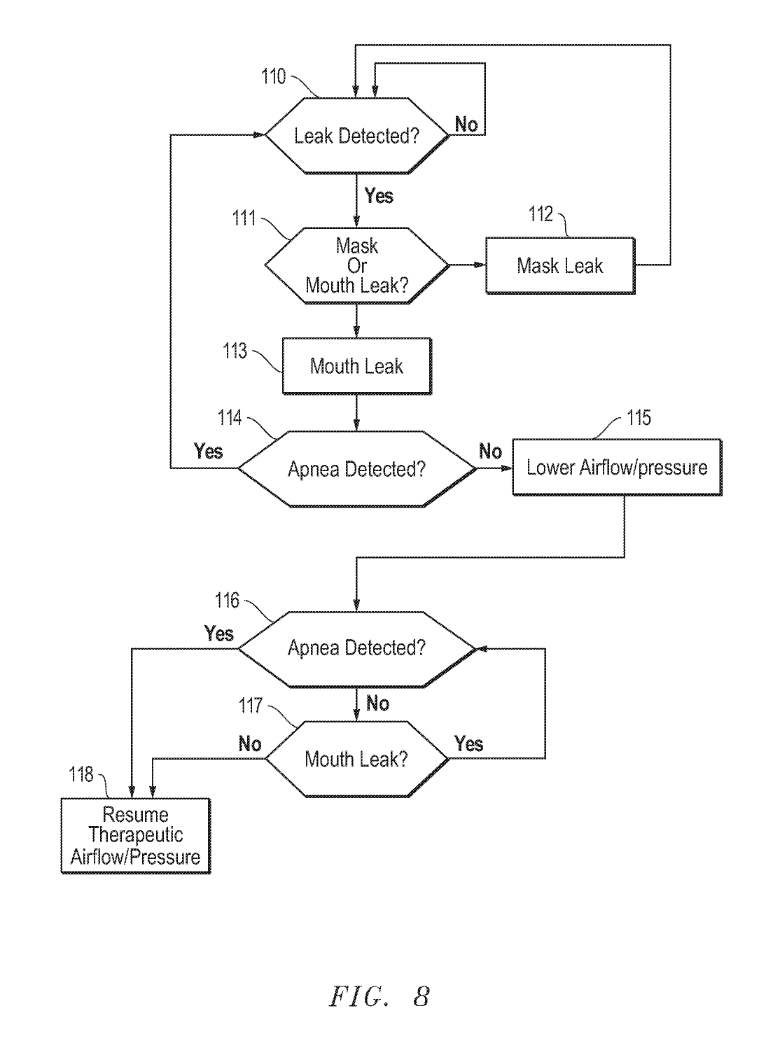

Now consider a patient using a positive airway pressure device with a blower, a control, pressure and/or airflow sensing, and a breathing circuit. Referring now to FIG. 8 which depicts a block diagram wherein each block represents a step or process in the process of determining the presence of a mouth leak. Block 110 detects and quantifies a leak. If no leak is present then block 110 continues monitoring for a leak. If a leak is detected then the quantified value is considered in block 111 to determine if it is a mask or mouth leak. If a mask leak is determined, then block 112 moves monitoring back to block 110. If a mouth leak is determined (block 113) then the system determines if an apnea or hypopnea event is present at block 114. If an apnea or hypopnea is present then the airflow, and thus pressure, is adjusted at block 115. Adjustment of airflow and pressure is preferably adjusted downward to prevent unnecessary drying of the patient's airway. Blocks 116 and 117 continue monitoring for apnea and hypopnea events and to determine if the mouth remains open. If an apnea or hypopnea occurs then the therapeutic pressures and airflow treatment resumes. Also, if the patient's mouth closes then the therapeutic pressures and airflow treatment resumes.

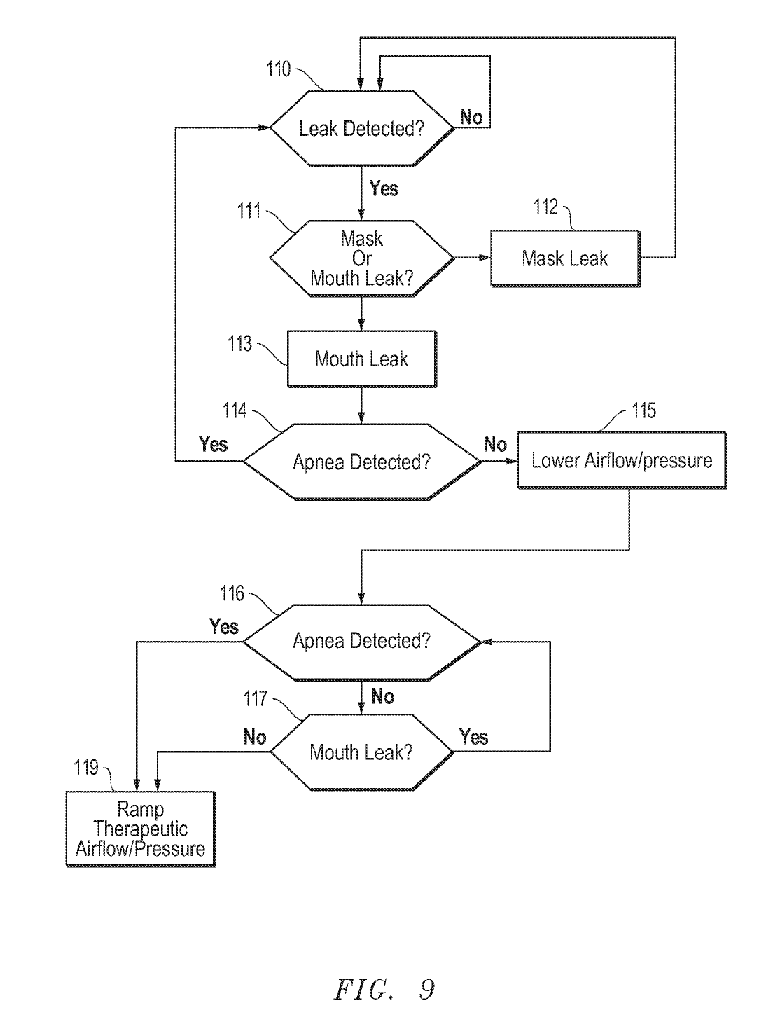

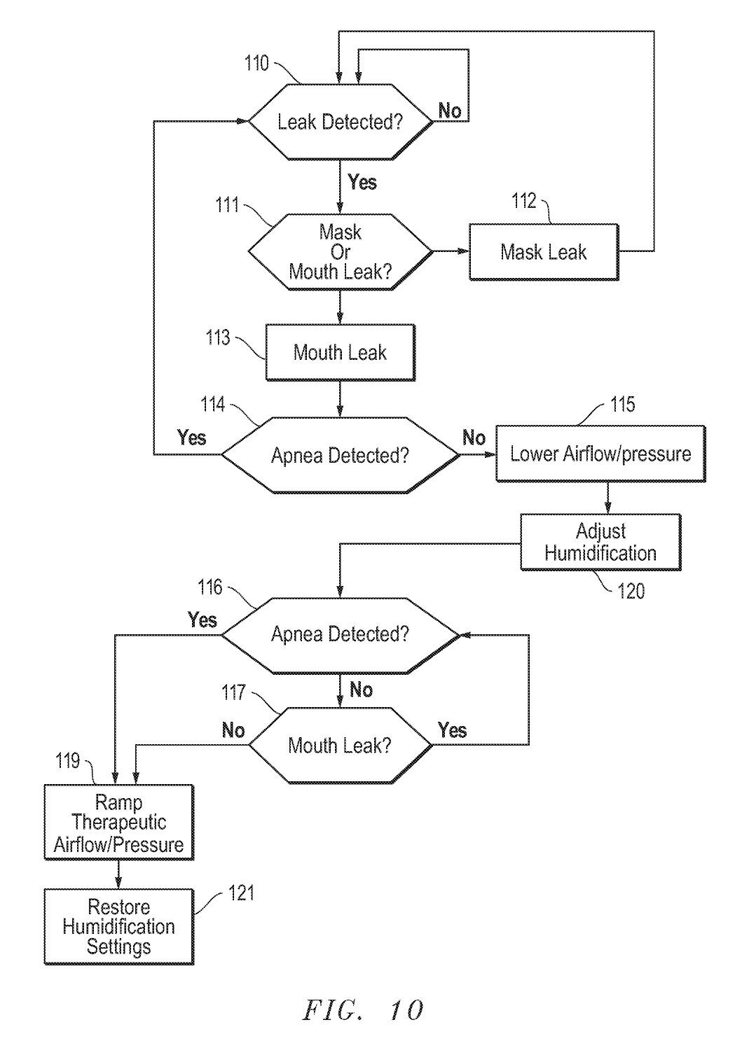

Referring now to FIG. 9, it is preferable in at least some instances to increase the therapeutic pressures and airflows to avoid patient arousals as depicted in block 121. As shown in FIG. 10, it may also be preferable to adjust the humidity levels at block 120 to aid in the prevention of patient airway drying and to restore the humidification levels at block 122.

In other embodiments of the present invention, the patient's delivery pressure is monitored over at least one sleep period. The optimal titration pressure from at least one previous sleep period is algorithmically determined and stored in memory for use during the next sleep period or for other future sleep periods. The stored value, or preferably a percentage of the stored value is used to determine the improved optimal and/or the starting pressure for the next or future sleep period. In one embodiment, the starting pressure at the onset of patient therapy is, for example, 50% of the stored optimal pressure. This enables the patient's optimal pressure to be determined more quickly resulting in improved sleep efficiency and less sleep related respiratory events. For example, if an optimal pressure from the previous sleep period is 14 cm/H2O then the starting pressure would be 7 cm/H2O. This enables a faster determination of the optimal pressure for that patient. In another embodiment, the starting pressure is predetermined. The stored pressure, or a percentage of the stored pressure, becomes the target pressure during a ramp-up sequence. This allows the patient to experience the benefit of a lower pressure at the beginning of a sleep period and allows for more linear and efficient ramping towards the target pressure. Since the target pressure is predetermined by the patients' own previous optimal pressure, the result is improved sleep efficiency and less sleep related respiratory events.

While the present system and method has been disclosed according to the preferred embodiment of the invention, those of ordinary skill in the art will understand that other embodiments have also been enabled. Even though the foregoing discussion has focused on particular embodiments, it is understood that other configurations are contemplated. In particular, even though the expressions “in one embodiment” or “in another embodiment” are used herein, these phrases are meant to generally reference embodiment possibilities and are not intended to limit the invention to those particular embodiment configurations. These terms may reference the same or different embodiments, and unless indicated otherwise, are combinable into aggregate embodiments. The terms “a”, “an” and “the” mean “one or more” unless expressly specified otherwise. The term “connected” means “communicatively connected” unless otherwise defined.

When a single embodiment is described herein, it will be readily apparent that more than one embodiment may be used in place of a single embodiment. Similarly, where more than one embodiment is described herein, it will be readily apparent that a single embodiment may be substituted for that one device.

In light of the wide variety of methods for detecting mouth leaks, the detailed embodiments are intended to be illustrative only and should not be taken as limiting the scope of the invention. Rather, what is claimed as the invention is all such modifications as may come within the spirit and scope of the following claims and equivalents thereto.

None of the description in this specification should be read as implying that any particular element, step or function is an essential element which must be included in the claim scope. The scope of the patented subject matter is defined only by the allowed claims and their equivalents. Unless explicitly recited, other aspects of the present invention as described in this specification do not limit the scope of the claims.

What is claimed is:

1. A method for administering continuous positive airway pressure comprising:
measuring respiratory air flow from a patient during the administration of continuous positive airway pressure to detect a mouth leak;
upon the detection of a mouth leak, decreasing the rate of said respiratory air flow to said patient if there is no indication of an apneic event.

2. A method for administering continuous positive airway pressure comprising:
measuring respiratory air flow from a patient during the administration of continuous positive airway pressure to detect a mouth leak;
upon the detection of a mouth leak, adjusting the humidity of said respiratory air flow to said patient if there is no indication of an apneic event.

3. A system for administering continuous positive airway pressure comprising:
a continuous positive airway pressure device coupled with a device for detecting the presence of a mouth leak;
wherein, upon said detection of said mouth leak, the rate of said respiratory air flow to said patient is decreased.

4. A system for administering continuous positive airway pressure comprising:
a continuous positive airway pressure device coupled with a device for detecting the presence of a mouth leak;
wherein, upon said detection of said mouth leak, the humidity of said respiratory air flow to said patient is adjusted.

5. A method for administering continuous positive airway pressure comprising:
measuring respiratory air flow from a patient as waveform as a function of time;
monitoring said waveform to determine if said patient is experiencing a mouth leak;
upon said determination of the presence of said mouth leak, decreasing the rate of said respiratory air flow.

6. A method for administering continuous positive airway pressure comprising:
measuring respiratory air flow from a patient in a waveform as a function of time;
establishing an approximate value of the root mean square voltage of said waveform during a period in which said patient is experiencing a mouth leak;
establishing an approximate value of the root mean square voltage of said waveform during a period in which said patient is experiencing an apneic event;
thereafter, monitoring said waveform using said approximate value of the root mean square voltage of said mouth leak to indicate the presence of a mouth leak and said approximate value of the root mean square voltage of said apneic event to indicate the presence of an apneic event; and
decreasing the rate of said respiratory air flow upon said indication of said mouth leak if there is no indication of said apneic event.

7. The method of claim 6, wherein after said decreasing said rate of said respiratory airflow, said respiratory airflow is increased to its original rate when there is no indication of said mouth leak.

8. The method of claim 6, wherein after said decreasing said rate of said respiratory airflow, said respiratory airflow is increased to its original rate when there is no indication of said apneic event.

9. The method of claim 6, wherein after said decreasing said rate of said respiratory airflow, said respiratory airflow is increased when there is no indication of said mouth leak.

10. The method of claim 6, wherein after said decreasing said rate of said respiratory airflow, said respiratory airflow is increased when there is no indication of said apneic event.

11. The method of claim 6, wherein after said decreasing said rate of said respiratory airflow, adjusting humidity of said respiratory airflow.

12. The method of claim 11, wherein after said humidity adjustment, said rate of said respiratory airflow is increased when there is no indication of said mouth leak and, thereafter, said humidity is readjusted.

13. The method of claim 6, wherein after said decreasing said rate of respiratory airflow, adjusting humidity of said respiratory airflow.

14. The method of claim 13, wherein after said humidity adjustment, said rate of said respiratory airflow is increased when there is no indication of said apneic event and, thereafter, said humidity is readjusted.

15. The method of claim 6, further establishing an approximate value of the root mean square voltage of said waveform during a period in which said patient's soft palate is partially blocking said patient's oral airway, monitoring said waveform using said approximate value of the root mean square voltage of said partial blockage to indicate said partial blockage, and decreasing said rate of respiratory air flow upon said indication of said partial blockage, if there is no indication of said apneic event.

16. The method of claim 15, wherein said decrease in said rate of said respiratory air flow is less upon said indication of said partial blockage than upon said indication of said mouth leak.

17. The method of claim 6, wherein said decrease in said rate of said respiratory air flow is accomplished in part through a release valve.

18. A method for administering continuous positive airway pressure comprising:
measuring respiratory air flow from a patient in a waveform as a function of time;
establishing an approximate first root mean square voltage of said waveform during a period in which said patient is experiencing a mouth leak;
establishing an approximate second root mean square voltage of said waveform during a period in which said patient is experiencing an apneic event;
thereafter, monitoring said waveform and decreasing the rate of said respiratory airflow to said patient when a root mean square voltage of said wave form approximates said first root mean square voltage, but not if such waveform approximates said second root mean square voltage.

19. A method for administering continuous positive airway pressure comprising:
measuring respiratory air flow from a patient in a waveform as a function of time;
establishing an approximate root mean square voltage of said waveform during a period in which said patient is experiencing an apneic event;
establishing an approximate root mean square voltage of said waveform during a period in which said patient is experiencing a mouth leak;
thereafter, monitoring said waveform and decreasing the rate of said respiratory airflow to said patient when a root mean square voltage of said wave form approximates said established root mean square voltage of said mouth leak, but does not approximate said root mean square voltage of said apneic event.

20. A method for administering continuous positive airway pressure comprising:
measuring respiratory air pressure from a patient in a waveform as a function of time;
establishing an approximate value of the root mean square voltage of said waveform during a period in which said patient is experiencing a mouth leak;
establishing an approximate value of the root mean square voltage of said waveform during a period in which said patient is experiencing an apneic event;
thereafter, monitoring said waveform using said approximate value of the root mean square voltage of said mouth leak to indicate the presence of a mouth leak and said approximate value of the root mean square voltage of said apneic event to indicate the presence of an apneic event; and
decreasing the rate of said respiratory air flow upon said indication of said mouth leak if there is no indication of said apneic event.

21. The method of claim 20, wherein after said decreasing said rate of said respiratory airflow, said respiratory airflow is resumed when there is no indication of said mouth leak.

22. The method of claim 20, wherein after said decreasing said rate of said respiratory airflow, said respiratory airflow is resumed when there is no indication of said apneic event.

23. The method of claim 20, wherein after said decreasing said rate of said respiratory airflow, said respiratory airflow is increased when there is no indication of said mouth leak.

24. The method of claim 20, wherein after said decreasing said rate of said respiratory airflow, said respiratory airflow is increased when there is no indication of said apneic event.

25. The method of claim 20, wherein after said decreasing said rate of said respiratory airflow, adjusting humidity of said respiratory airflow.

26. The method of claim 25, wherein after said humidity adjustment, said rate of said respiratory airflow is resumed when there is no indication of said mouth leak and, thereafter, said humidity is restored to its original value.

27. The method of claim 20, wherein after said decreasing said rate of said respiratory airflow, adjusting humidity of said respiratory airflow.

28. The method of claim 27, wherein a after said humidity adjustment, said rate of said respiratory airflow is resumed when there is no indication of said apneic event and, thereafter, said humidity is restored to its original value.

29. A system for administering continuous positive airway pressure comprising:
a blower fluidly connected to a hose which is fluidly connected to a mask, wherein said blower is configured to blow air through said hose through said mask to a patient;
an air flow sensor configured to monitor the rate at which said air flows through said hose;
a monitor connected to said sensor, wherein said monitor depicts air flow from a patient in a waveform as a function of time;
wherein an approximate value of the root mean square voltage of said waveform is established during a period in which said patient is experiencing a mouth leak, an approximate value of the root mean square voltage of said waveform is established during a period in which said patient is experiencing an apneic event, and, thereafter, said waveform is monitored using said approximate value of the root mean square voltage of said mouth leak to indicate the presence of a mouth leak and said approximate value of the root mean square voltage of said apneic event to indicate the presence of an apneic event, and
said respiratory air flow is decreased upon said indication of said mouth leak provided there is no indication of said apneic event.

30. The system of claim 29, wherein after said decreasing said rate of said respiratory airflow, said respiratory airflow is resumed when there is no indication of said mouth leak.

31. The system of claim 29, wherein after said decreasing said rate of said respiratory airflow, said respiratory airflow is resumed when there is no indication of said apneic event.

32. The system of claim 29, wherein after said decreasing said rate of said respiratory airflow, said respiratory airflow is increased when there is no indication of said mouth leak.

33. The system of claim 29, wherein after said decreasing said rate of said respiratory airflow, said respiratory airflow is increased when there is no indication of said apneic event.

34. The system of claim 29, wherein after said decreasing said rate of said respiratory airflow, adjusting humidity of said respiratory airflow.

35. The system of claim 34, wherein after said humidity adjustment, said rate of said respiratory airflow is resumed when there is no indication of said mouth leak and, thereafter, said humidity is restored to its original value.

36. The system of claim 29, wherein after said decreasing said rate of said respiratory airflow, adjusting humidity of said respiratory airflow.

37. The system of claim 34, wherein a after said humidity adjustment, said rate of said respiratory airflow is resumed when there is no indication of said apneic event and, thereafter, said humidity is restored to its original value.
